(19) United States
(12) Patent Application Publication
(10) Pub. No.: US 2011/0123470 A1
(43) Pub. Date: May 26, 2011

(54) NATURAL BIOACTIVE COMPOUNDS

(75) Inventors: Liming Yan, Argyll (GB); Andrew Mearns Spragg, Argyll (GB); Karen Jukes, Argyll (GB)

(73) Assignee: Aquapharm Bio-Discovery Ltd., Oban (GB)

(21) Appl. No.: 12/598,744

(22) PCT Filed: May 2, 2008
(86) PCT No.: PCT/GB2008/001544

§ 371 Date: Oct. 25, 2010

(30) Foreign Application Priority Data
May 4, 2007 (GB) 0708611.9
Nov. 30, 2007 (GB) 0723451.1

(51) Int. Cl. A61K 8/49; A01N 43/08; A61K 31/341; A61K 47/22; C12P 17/04; C07D 307/02; A61P 31/10; A01P 3/00; A01P 1/00; A01P 15/00; A61P 31/12; A61P 31/04; A23L 3/3544; A23L 1/226; A23L 2/00; A23D 9/013; C12N 1/00

(52) U.S. Cl. 424/62; 514/473; 514/772; 424/70.1; 435/126; 549/479; 426/335; 426/536; 426/590; 426/531; 435/254.1

(57) ABSTRACT

The present invention relates to butenolide compounds having cytoprotection such as antioxidant, anti-inflammatory and/or antifungal properties, and which are derived from the marine fungus Aureobasidium.

### INTRODUCTION

The present invention relates to butenolide compounds having cytoprotection such as antioxidant, anti-inflammatory and/or antifungal properties, and which are derived from the marine fungus Aureobasidium.

### BACKGROUND TO INVENTION

Butenolides or furanones are a class of heterocyclic lactones that contain four carbons. The majority of known butenolides have reportedly been derived from plant fruits (Rouseff, Leahy et al. 1995). However, some bacteria and fungi have also been reported to be capable of producing butenolides. For example, butenolides are precursors of gamma-butyrolactones which are considered to be involved in quorum-sensing cell-cell signalling systems in Streptomyces species (Dunny and Leonard, 1997). Gamma-butyrolactones play an important role in regulating morphogenesis, sporulation, differentiation and secondary metabolism, importantly antibiotic production in Streptomyces (Braun et al., 1995; Takano et al., 2000; Dunny and Leonard, 1997; Kato et al., 2007; Takano, 2006). Streptomyces have also been cited at being able to produce butenolide antibiotic compounds such as butalactin (Franco et al., 1991). In addition, some Gram-negative bacterial species, for example, Pseudomonas aureofaciens has also been reported to produce (Z)-4-hydroxy-4-methyl-2-(1-hexenyl)-2-butenolide and (Z)-4-hydroxymethyl-2-(1-hexenyl)-2-butenolide. Although micro-organisms are able to produce various butenolide compounds, a compound according to formula (I) as defined herein after, has never to the best of our knowledge, been reported to be produced by any bacterial or fungal species.

A series of antifungal cyclic depsipeptides named aureobasidins have been isolated from supernatant of the species Auresbasidiam sp. (Ikai, Takesako et al. 1991; Yoshikawa, Ikai et al. 1993; In, Ishida et al. 1999). Currently aureobasidins are the only antifungal compounds isolated from the genus Aureobasidium. All of the compounds in this chemical family share a similar cyclic depsipeptide structure. Butenolides have not been reported to be produced by Aureobasidium sp.

Many butenolides and gamma-butyrolactones exhibit very intense and pleasant fruity aroma (Buttery and Ling, 1998). It has been documented that coconut aldehyde (5-pentyl-4,5-dihydro-2(3H)-furanone), as well as its analogues such as 5-butyl-4-methyl-4,5-dihydro-2(3H)-furanone (whiskey lactone) and 5-isobutyl-3-methyl-4,5-dihydro-2(3H) furanone give off pleasant fragrances similar to coconut (Sinha et al, 2004). However, no furan-2(5H)-one compound has been previously reported to give this type of fragrance. Testing of the flavour or fragrance of the compound 5-hexylfuran-2(5H)-one does not appear to have been documented.

Japanese patent No. 2005-35929 to Kanebo Ltd and Soda Aromatic Co. Ltd., generally describes an antifungal agent containing a gamma-lactone (compound A):

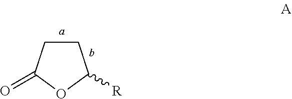

where the alkyl group R stands for one to twelve carbon alkyl group, and bonds “a” and “b” can be a single or double bond. Several active antifungal ingredients are claimed by Kanebo, however all apart from one compound are saturated butenolides (bonds “a” and “b” are both single bonds), the other compound being 5-methyl-2(3H)-furanone (bond “a” is a single bond and bond “b” is a double bond). A further compound, 5-methylfuran-2(5H)-one is mentioned within a list of the already mentioned compounds (R is methyl, bond “a” is a double bond and bond “b” is a single bond), but no data on antifungal activity is presented for that methyl-butenolide compound. Furthermore, the disclosure does not describe any of the compounds as having antioxidant, cytoprotective or anti-inflammatory activities.

The promotion of inflammatory conditions and the initiation of the innate immune response requires expression of a great variety of important cytokines, one of which is TNF-α. Nuclear factor kappa B (NF-κB) is one of the principal inducible transcription factors which control the transcription of those cytokine genes so that it plays a pivotal role in the mammalian innate immune response (Herfarth, Brand et al. 2000; Nichols, Fischer et al. 2001). Consistent with this role, incorrect regulation of NF-κB has been linked to cancer, autoimmune diseases, septic shock, viral infection and improper immune development. Therefore, NF-κB has been implicated in processes of anti-inflammatory targets (Kim, Jeong et al. 2005; Moussaieff, Shohami et al. 2007). In addition, inhibitor kappa B kinase β (IKKβ) phosphorylates the IκB proteins, leading to their degradation and the subsequent activation of gene expression by NF-κB (Karin and Delhase, 2000; Yamamoto, et al., 2000). Therefore, the IKKβ activity is also involved in the regulation of the inflammatory response.

There are few reports that have compared the effects of various small γ-lactone compounds on the induction or inhibition of NF-κB in mammalian cells. Among some complicated lactones, sesquiterpene lacone is a potent anti-inflammatory molecule whose mode of action was proposed to inhibit activation of NF-κB (Lyss, Knorre et al. 1998; Koch, Klaas et al. 2001), the alpha-methylene-gamma-lactone moiety of the sesquiterpene lactones was required for inhibitory activity (Hall, Lee et al. 1979); clastolactacystin beta-lactone could also inhibit translation of NF-κB (Ding, Fischer et al. 1998). However, brefeldinA could activate NF-κB (Lin, Boller et al. 1998). Anti-inflammatory activity or any effects on NF-κB biosynthesis by compounds on basis of the formula A have not been reported.

An object of the present invention is to provide further butenolide compounds having cytoprotective, such as antioxidant or glutathione elicitation and/or antifungal activity and/or anti-inflammatory activity.

A further object of the present invention is to provide such compounds having a pleasant odour and/or flavour.

A still further object of the present invention is to provide a method to produce natural gamma-alkyl butenolides including desired isomeric forms thereof (e.g. R form) such as 5-alkylfuran-2(5H)-one from a marine Aureobasidium sp. isolate.

### SUMMARY OF INVENTION

According to a first aspect of the present invention, there is provided the use of a compound according to formula (I),

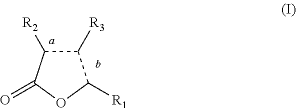

wherein,

R₁ is a C₁-C₄₀ alkyl group,

R₂ and R₃ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone, the dashed lines a and b are independently single or double bonds, but not both together double bonds, in the manufacture of an antifungal, cytoprotective and/or anti-inflammatory agent.

A suitable cytoprotective response may be one which is capable of inducing intracellular anti-oxidants such as gluthathione, e.g. through activation of a mammalian anti-oxidant response element (ARE) gene battery. A suitable anti-inflammatory agent may be one which is capable of suppressing NF-kB response.

Alternatively stated, an antifungal cytoprotective and/or anti-inflammatory compound according to formula (I) is provided.

Preferred antifungal compounds of formula (I) of the present invention include those wherein a is a double bond and R₁ is a C₆-C₂₈, preferably a C₆-C₂₀ alkyl group.

Preferably R₂ and/or R₃ are H.

Many of the subject compounds display pronounced and distinctive fragrance and flavour properties, which is seen as a further benefit.

According to a second aspect of the present invention, there is provided a compound according to the above described formula (I) for use as a fragrance and/or flavouring agent. Alternatively stated, a fragrance and/or flavouring agent according to formula (I) is provided.

In particular, the compound according to formula (I), wherein R is n-hexyl and has the optical rotation [α]²⁰_{D} −107.3 (c=1.18, CHCl₃); {lit. [α]²⁰_{D} −84.1 (c=1.01, CHCl₃)}¹ and desirably displays a coconut fragrance and flavour.

The applicant of the present invention has also identified that the subject compounds may alternatively or further exhibit cytoprotective, such as antioxidant and/or anti-inflammatory properties.

The compounds of the present invention are shown as cyclic structures. However, without wishing to be bound by theory, the active form may be an uncyclised form.

The formula (I) compounds can be provided in compositions, either alone or in combination with other cytoprotective, antifungal and/or anti-inflammatory or other active ingredients or with carriers, as may be determined by the person skilled in the art.

The alkyl group R₁ of formula (I) may be branched or unbranched, for example typical branched alkyl groups include iso-propyl, iso-butyl, sec-butyl, tert-butyl, 3-methylbutyl, 3,3-dimethylbutyl and variations, including isomers thereof. Preferred alkyl groups for the antifungal compounds are straight chain.

Further, R₁ may be substituted with a group selected from alkenyl, alkynyl, aryl, heteroaryl, carboxy, alkyloxycarbonyl, hydroxyl, amino, nitro, alkyloxy, alkylthio, formyl, cyano, carbamoyl, halo or a ketone.

Generally, the alkyl and alkenyl groups stated herein may be straight chain, branched chain or cyclic. Alkynyl groups may be straight chain or branched chain.

Halo includes fluoro, chloro, bromo and iodo.

A preferred compound for use as an antifungal, cytoprotective (antioxidant) anti-inflammatory antibacterial and/or flavourant/fragrance is 5-hexylfuran-2(5H)-one, (also named as 5-hexyl-5H-furan-2-one or 5-hexyl-2(5H)-furanone). This compound shows particularly potent anti-fungal activity at low concentrations e.g. at a minimum inhibitory concentration (MIC) of 1 μg/ml or less or 3 μg/ml or less, in particular against Candida albicans Pityrosporum ovale and Malassezia furfur. The above compound is also shown herein to be anti-inflammatory and shows particularly potent suppression of NF-kB.

Fungi which may be targeted by the subject compounds of formula (I) include Trichophyton species, such as Trichophyton rubrum, Aspergillus species, such as Aspergillus fumigatus, Candida species, such as Candida albicans, Pityrosporum species, such as Pityrosporum ovale and Malassezia species, such as Malassezia furfur. Other fungi may be targeted by the subject compounds, such as Trichophyton rubrium.

It will be appreciated that the compounds of formulae (I) and (II) for use in the present invention may be applied at a concentration or dose depending on the purpose to which the compound is being put. In particular, when used in a method for killing fungi, the amount of subject compound in a composition against the target fungus is of the order of less than or equal to 100 μg/ml, such as 50 μg/ml, preferably 1-20 μg/ml, most preferably 1-10 μg/ml e.g. 1-5 μg/ml.

It will be appreciated that the compounds of formulae (I) and (II) for use in the present invention may exist in various stereoisomeric forms and the compounds for use in the present invention as hereinbefore defined include all stereoisomeric forms and mixtures thereof, including enantiomers and racemic mixtures. The present invention includes within its scope the use of any such stereoisomeric form or mixture of stereoisomers, including the individual enantiomers of the compounds of formula (I) as well as wholly or partially racemic mixtures of such enantiomers. More preferential is the use of the form shown in FIG. 1a.

A marine Aureobasidium sp. strain AQP1639, which was isolated from marine sediment, produces antifungal and antioxidant compounds according to formulae (I) and (II) when grown in a medium enriched with carbohydrates, but limited nitrogen sources.

The marine Aureobasidium sp. strain AQP1639 was deposited by the Applicant in December 2006, in accordance with the Budapest Treaty, at the CABI Bioscience UK Centre (IMI) and having accession number IMICC No. 394867.

The compounds for use in the present invention may be prepared using reagents and techniques readily available in the art, such as synthetic organic chemistry methods, and as described hereinafter.

An example procedure, which may be applied for bulk manufacture of the subject compounds, uses internal cyclisation of hydroxylated unsaturated fatty acids.

This cyclisation may be achieved using an isolated enzyme, such as an esterase enzyme.

According to a further aspect of the present invention, there is provided a method of preparing a compound according to the present invention, comprising the steps:

i) providing an Aureobasidium sp. fungus, and culturing in a suitable culture medium under conditions and for an appropriate time period suitable for production of said compound or compound precursor; and

ii) recovering said compound, from the resultant culture.

Preferably, the Aureobasidium sp. is a marine species.

Most preferred, is the Aureobasidium sp. strain deposited in accordance with the Budapest Treaty at the CABI Bioscience UK Centre IMI on the Dec. 20, 2006 and having accession number IMICC No. 394867, or mutant or variant thereof having the property of producing the subject compounds.

The culturing may be performed according to any suitable method available to the skilled person, and includes fermentation of the stated fungal species.

The preparation of the stated compounds may also be achieved using an enzyme or mixture of enzymes obtained from the Aureobasidium sp. which enable the chemical reactions to proceed to form the final desired compound, optionally including intermediates of the desired compound. For example, an enzyme or mixture of enzymes may be used in a cyclisation reaction to form the final cyclised compound, e.g. an esterase enzyme. The enzyme or mixture of enzymes may be used to form an intermediate product, such as a non-cyclised compound, which through further chemical treatment, may be converted to the final desired compound, e.g. by cyclisation. An example of further chemical treatment includes exposure of the intermediate compound to acidic and/or alkaline conditions, e.g. by use of an inorganic acid or alkali. Acids include sulphuric and hydrochloric acid. Alkalis include group I or group II metal hydroxides, e.g. sodium hydroxide.

The amount of produced compound according to formula (I) and/or (II) may be enhanced/optimised by culturing the Aureobasidium sp. and ascertaining the level of produced compound, followed by altering the culturing conditions and remeasuring the level of compound. This procedure may be repeated until optimised conditions are arrived at.

The procedure of optimisation may involve altering the culture conditions by changing the ratio of carbon to nitrogen in the culture medium.

An optimised procedure includes use of a culture medium enriched in an amount of carbon, e.g. carbohydrate, and limited in an amount of nitrogen, so that a high carbon to nitrogen ratio is obtained. This may be achieved by using a suitable growth substrate comprising a greater amount of carbohydrate than nitrogen components, but excluding from the substrate further addition of a nitrogen source such as yeast extract or peptone. An example is a media comprising 24 g Potato Dextrose base in 1 L seawater.

Suitable carbon to nitrogen ratios include, 20:1, preferably 15:1, desirably 11:1.

For example, a starch source is suitable for providing carbohydrate, and seawater for providing a restricted quantity of nitrogen. A desirable method includes pre-culturing in Potato Dextrose Agar with natural sea-water for an appropriate length of time, e.g. two to three weeks. Following pre-culture, the method comprises inoculation of Potato Dextrose Broth in sea-water and culturing for an appropriate length of time, e.g. three to four weeks.

Recovering the subject compounds may require extraction of the culture supernatant by any suitable solvent, e.g. an organic solvent such as ethyl acetate or the like, followed by alkalinisation of the extract, using e.g. an inorganic base, such as sodium hydroxide, which may be provided as an aqueous solution.

Suitable pH conditions range from 9 to 11, for example 10 to 11.

Addition of a polar solvent such as an alcohol, e.g. methanol, followed by extraction with a non-polar solvent, e.g. hexane, provides a crude desired product, which may then be purified further using techniques available to the skilled person such as chromatography.

The invention provides a simple way to produce natural butenolide compounds with useful pharmaceutical properties, including, but not limited to, antimicrobial properties, antioxidant, anti-inflammatory, and/or anti-cancer properties.

Antimicrobial includes antifungal, antibacterial, and/or anti-viral, including activity against pathogenic and non-pathogenic organisms. Preferably, antimicrobial refers to antibacterial.

Particular conditions which may be treated include acne, dandruff, nail fungus, cancer, inflammatory and autoimmune diseases, septic shock, viral infection and improper immune development, stress, cytokines, free radicals, ultraviolet irradiation and bacterial or viral antigens synaptic plasticity, memory and anti-inflammatory targets and regulating the immune response to infection. Other fungal conditions caused by Candida albicans, Malassezia furfur and Trichophyton rubrum may also be treated with the compounds described herein.

The present invention further provides a treatment or prophylaxis of a disease, pathology or condition recited herein comprising administering a compound recited herein to a patient in need thereof.

The patient is typically an animal, e.g. a mammal, especially a human.

The subject compounds may be applied topically to the patient, e.g. applied to the skin.

The applicant has found that in particular, the subject compounds display low toxicity in mammalian cells, e.g. rat and human.

The applicant has further observed that the subject compounds have differential effects on rat and human cells providing differential cytotoxicity effects, suggesting usefulness of those compounds in anticancer therapy.

The compounds are also useful in cosmetic and cosmeceutical applications, e.g. related to personal care, including, but not limited to, skin anti-ageing, skin toning/smoothing, altering skin pigmentation, such as affecting melanin levels, e.g. for skin whitening, and dermal and hair treatments.

Use also as food additives, flavouring, preservatives and nutritional supplements is provided.

For use according to the present invention, the compounds or physiologically acceptable salt, ester or other physiologically functional derivative thereof described herein may be presented as a pharmaceutical formulation, comprising the compound or physiologically acceptable salt, ester or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers, which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

Preparations for personal care and cosmeceutical uses can be provided according to methods available to those of skill in the art. For example, the active compounds may be presented for topical uses in preparations such as skin cremes (e.g. facial cremes), washes (e.g. facial washes), rinses, shampoo, conditioners, hair dyes, pomades, mousses, and the like. The odour additive, preservative, or antioxidant effects of the compounds provides particular suitability for these uses. The skilled person will be able to provide the remaining components of such preparations according to the use. For example, typical ingredients may include water, alcohols, wetting agents, surfactants, oils, waxes, gelling agents, colourants and the like.

Additional uses include providing the active compounds, either alone or included within a preparation, as a food additive to provide antioxidant properties, anti-spoilage, preservative and/or flavouring to food and/or beverage. The active compounds may also be provided in suitable form, e.g. tablet form, as a nutritional supplement.

Usefully, the compounds described herein may be provided as intermediates or substrates for the preparation of further compounds, particularly biologically active compounds, such as those having the above-noted properties, in particular personal care, cosmetic, food and nutritional applications.

The present invention will now be described with reference to the following non-limiting examples and drawings.

### BRIEF DESCRIPTION OF THE DRAWINGS

FIG. 1 is the chemical structure of 5-hexylfuran-2(5H)-one (named P1639C);

FIG. 1a shows the optical rotation of 5-hexylfuran-2(5H)-one (named P1639C);

FIG. 2a is a graph showing elicitation of ARE-driven luciferase activity by tBHQ;

FIG. 2b is a graph showing elicitation of ARE-driven luciferase activity by 5-hexylfuran-2(5H)-one (P1639C);

FIG. 2c is a graph showing elicitation of ARE-driven luciferase activity by 4-decanolide;

FIG. 2d is a graph showing elicitation of ARE-driven luciferase activity by 2(5H)-furanone.

FIG. 3a shows the COSY nmr correlations for the compound P1639C;

FIG. 3b shows the HMBC nmr correlations for the compound P1639C;

FIG. 4a shows the proton-NMR spectrum for the compound P1639C;

FIG. 4b shows the ¹³C-NMR spectrum for the compound P1639C;

FIG. 5 is the graph resulting from LR Mass spectrometry for the compound P1639C;

FIGS. 6a-c are three graphs resulting from toxicology assay carried out using an in vitro human hepatocytes model where human hepatocytes were exposed to varying concentrations of compound P1639C (FIG. 6c) as well as tamoxifen (FIG. 6a) and chlorpromazine (FIG. 6b) for three hours.

FIGS. 7a & b are two charts showing the ability of compound P-1639 to suppress NFκB anti-inflammatory activity using an in vitro mammalian cell model. Cells were exposed to varying concentrations of P1639C as well as chemical analogues of P1639C.

FIG. 7a Anti-inflammatory activity and cytotoxicity of p1639C at various concentration. The anti-inflammatory assay was examined using the inhibition of NF-κB activity. The anti-inflammatory activity of p1639C showed a significant dose-dependant response. A concentration of 0.5 mM of p1639C could almost fully inhibited the NF-κB mediated inflammatory response, and 0.02 mM could inhibit 50%. In addition, there was no significant cytotoxicity was observed against the testing cells. Cell only: without inflammatory elicitation; TNF-α: inflammation was elicited by TNF-α and used as 100% inflammatory response (negative control); SEAP: an known inflammatory response enhancer; Bay-11: an known anti-inflammatory agent used as the positive control.

FIG. 7b. Comparison of NF-κB inhibition activity between p1639C and other known butenolides with a similar structure. The results shows that p1639C is the only active compound among them. All the tested butenolides did not show significant cytotoxicity. All the known butenolides tested were also at a concentration of 0.05 mM.

FIGS. 8a-d show the anti-oxidant elicitation effects by a known antioxidant tert-butylhydroquinone (tBHQ, a), p1639C (5-hexyl-2(5H)-furanone, b), 4-decanolide (5-hexyl-2(3H)-furanone, c) and 2(5H)-furanone (d).

FIG. 9 shows the timecourse of Electron Paramagnetic Resonance (EPR) signal development with menadione and pyrogallol in the presence of different concentrations of ascorbic acid or p1639C compound.

### DETAILED DESCRIPTION OF THE INVENTION

### Examples

The following examples are given by way of illustration of the present invention and should not be considered to limit the scope of the present invention.

### I. Production of Natural R Isomer Gamma-Alkyl Butenolides

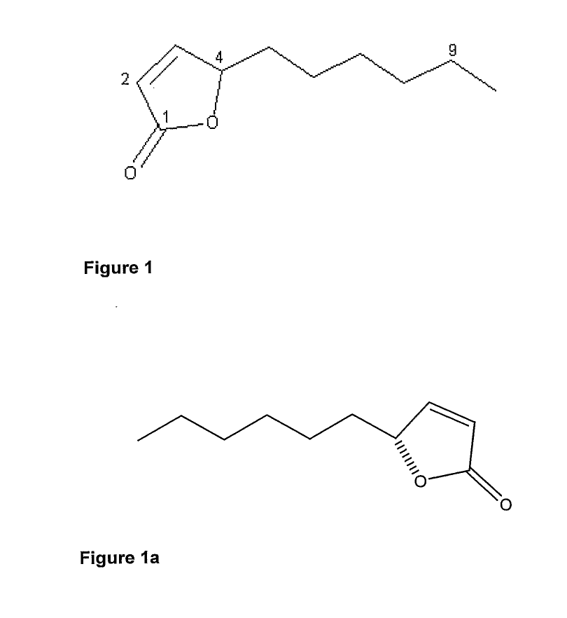

The optimized protocol for the production of the gamma-alkyl butenolides involves culturing the marine Aureobasidium sp. strain, AQP1639 in a carbohydrate enriched media (e.g. 24 g potato dextrose medium in 1 L natural seawater) prepared with natural seawater. Production of the gamma-alkyl butenolides by AQP1639 also requires agitated planktonic suspension cultivation with adequate oxygen supply, and alkalisation of the ethyl acetate extract from crude natural product. The yield of the gamma-alkyl butenolides, 5-hexylfuran-2(5H)-one (named P1639C; FIG. 1), produced under non-optimised culture conditions is approximately 10˜15 mg/L.

AQP1639 was pre-cultured in Potato Dextrose Agar (PDA, Oxoid) prepared with natural sea water. The growth medium was autoclaved at 121° C. for 15 min before plates were made. AQP1639 was inoculated on a PDA seawater plate and cultivated for two to three weeks until darkly pigmented arthroconidia became obvious. The pre-grown colonies were used to inoculate Potato Dextrose Broth (PDB, Oxoid) which was also prepared using natural seawater from the same source, followed by shaken flask cultivation at 30° C. at a speed of 220 rpm for 20 days. The cultivation was carried out until a visible black biofilm was established at the air/liquid interface on the flask wall, which usually took three to four weeks. Dark oil could be seen at air/liquid interface at this stage. The ethyl acetate extract of the culture supernatant, which showed antimicrobial activity, was dried and then alkalinised using 0.5M NaOH water solution in room temperature (20° C.˜24° C.) until the oil-like material dissolved in water completely. MeOH was then added to the alkalinised solution and mixed thoroughly, followed by hexane extraction. The hexane extract was then evaporated down completely and again reconstituted in 2 ml hexane. The hexane reconstitute was then loaded to silica Sep-Pak® cartridge to carry out antifungal activity guided fractionation using 100% hexane, 90% hexane:10% EtoAc and 80% hexane:20% EtoAc. The active fractions were pooled and reconstituted in EtoAc, then further purified using C18-HPLC and isocratic 70% MeOH:30% H₂O. The active fractions were pooled and the structure was characterised using NMR spectroscopy and mass spectrometry.

### II. Characterisation of P1639C

P1639C showed a LRMS of m/z 191.2 (M+Na)⁺. Careful analysis of the NMR data (Table 2) suggested a molecular formula of C₉H₁₄O₂. With an unsaturation number of three suggested the presence of one ring in the system. Correlations from COSY and HMBC (FIG. 3) yielded a known butenolide-type, 5-hexylfuran-2(5H)-one (Mukku, 2000) compound. This compound produced a pleasant fragrance similar to coconut-oil, and is an analogue of the known 2,6-dimethyl-5-oxo-heptanoic acid, a widely used flavour in food stuffs, alcoholic beverages, perfumery and pharmaceutical products (Sinha, 2004). Optical rotation measurements was recorded using a Perkin Elmer, Model 343 Polarimeter at 589 nm.

TABLE 1
¹H-NMR at 400 MHz and ¹³C at 100 MHz in CDCl₃ for P1639C

| | ¹³C δ/ppm, | | | |
|---|---|---|---|---|
| C # | mult | ¹H δ/ppm, mult, J (Hz) | COSY | HMBC |
| 1 | 164.78 (s) | | | H-2, H-3, |
| 2 | 121.6 (d) | 5.96 (1H, 9.6, 2.4) | H-3 | H-4 |
| 3 | 145.2 (d) | 6.83 (1H, 10.0, 0.4) | H-2, H-5 | H-2, H-4, |
| | | | | H-5 |
| 4 | 78.2 (d) | 4.38 (1H, m) | H-5, H-6A, | |
| | | | H6B | |
| 5 | 29.6 (d) | 2.29 (2H, m) | | |
| 6 | 35.0 (t) | A: 1.75 (1H, m) | H-6B, H-4 | H-3, H-5, |
| | | | | H-7A, H-7B |
| | | B: 1.60 (1H, m) | H-6A, H-4 | |
| 7 | 24.7 (t) | A: 1.75 (1H, m) | H-7B | H-4 |
| | | B: 1.60 (1H, m) | H-7A | |
| 8 | 31.7 (t) | 1.27 (2H, m) | | H-9, H-7A, |
| | | | | H-7B |
| 9 | 22.7 (t) | 1.27 (2H, m) | H-8, H-10 | H-10 |
| 10 | 24.2 (q) | 0.85 (3H, t, 7.2, 6.8) | H-9 | H-8, H-7A, |
| | | | | H-7B |

### III. Antioxidant Elicitation Assay (A.R.E)

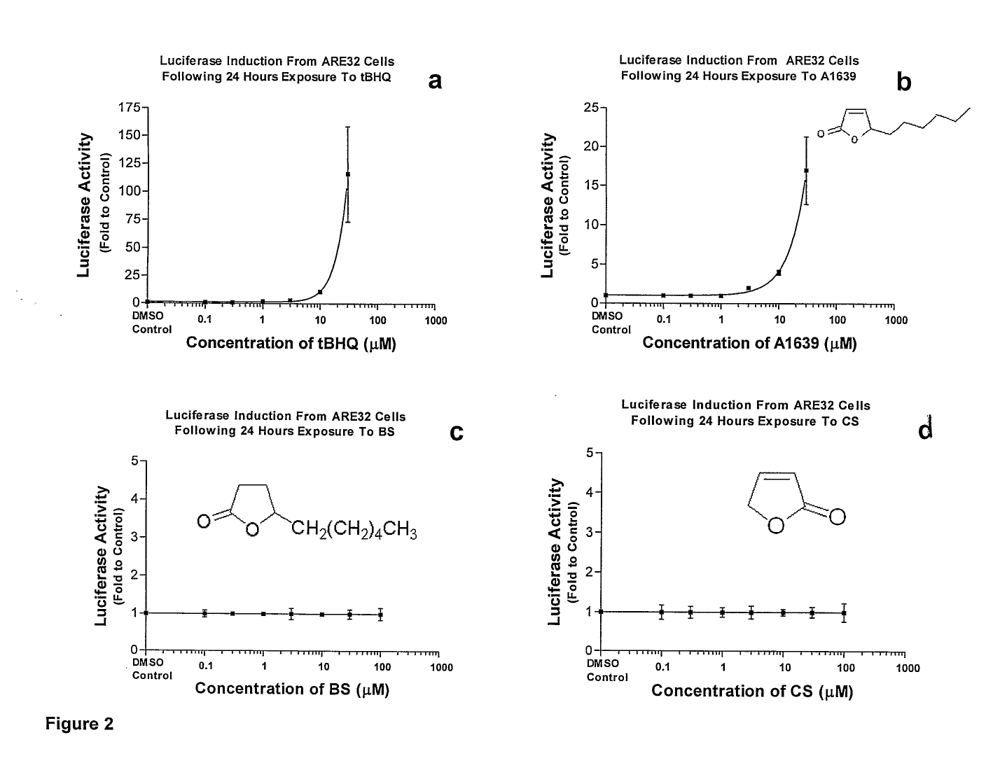

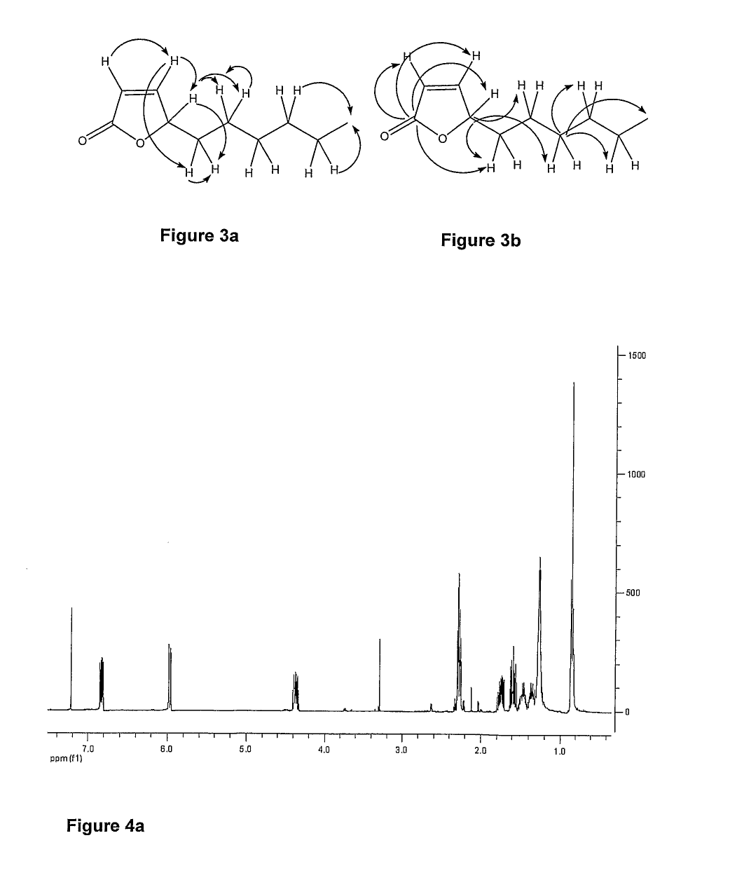

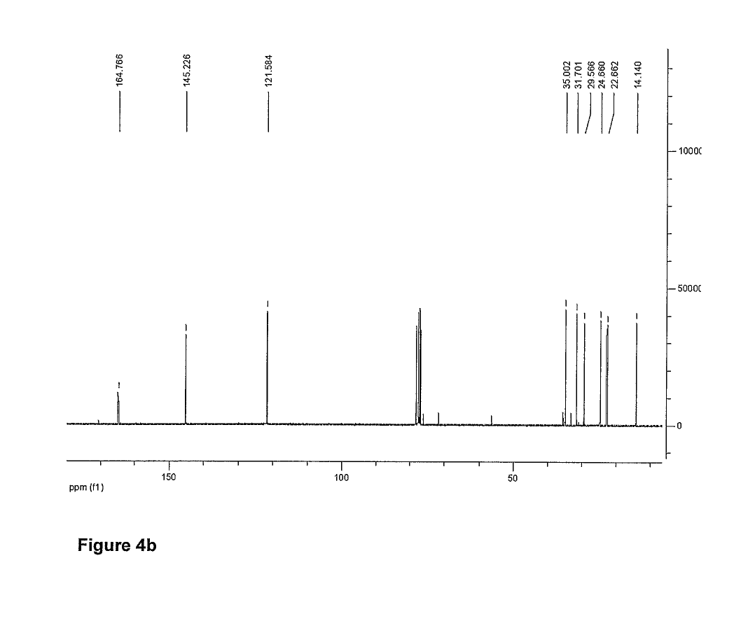

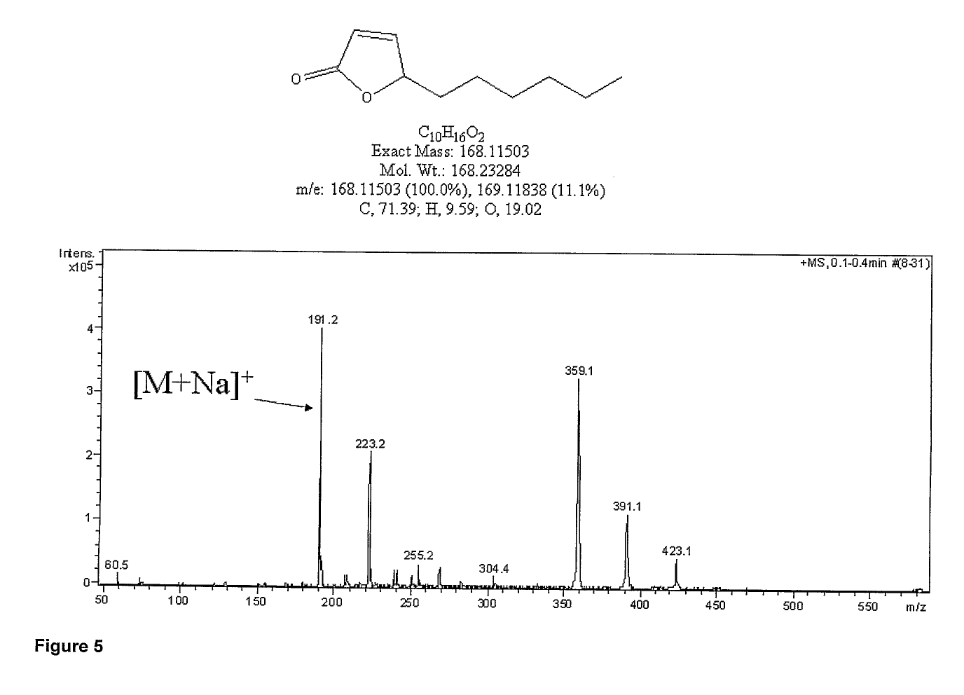

An antioxidant assay was carried out using an antioxidant reporter cell line to determine if P1639C upregulated the protective anti-oxidant gene battery which is under control of the anti-oxidant response element (ARE). The cell line ARE32 was incubated with eight concentrations of P1639C and the positive control tert-butylhydroquinone (tBHQ) for 24 hours, and the luciferase activity measured (Luciferase Assay System, Promega). P1639C enhanced induction of ARE-driven luciferase activity up to 18-fold (at a concentration of 30 μM) (FIG. 2b) compared with normal cells without any oxidative induction. Thus, the butenolide P1639C, produced by AQP1639, showed potent antioxidant elicitation activity.

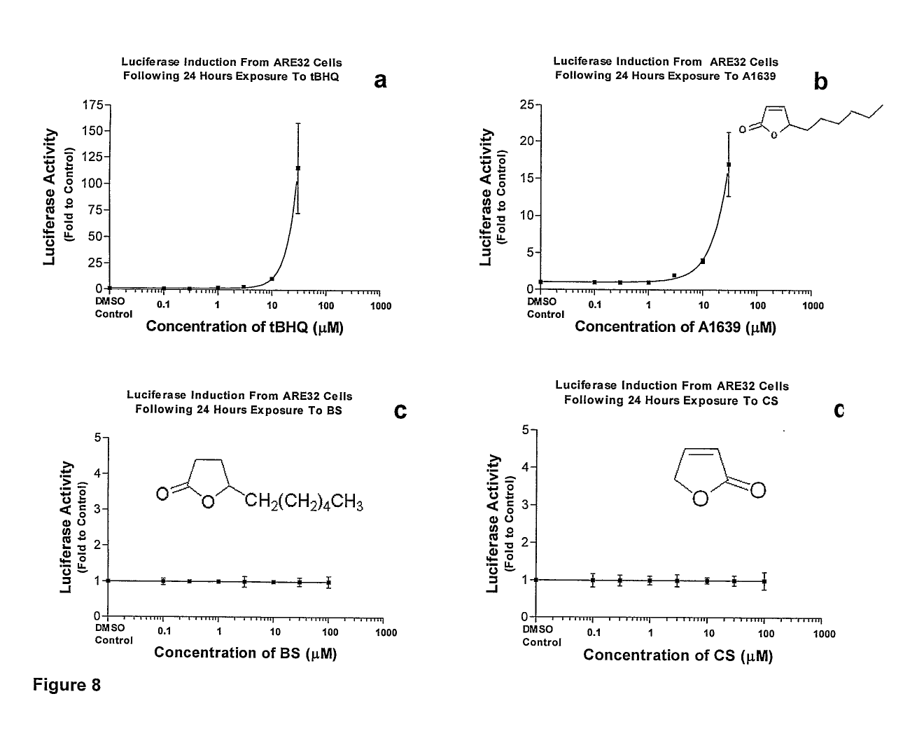

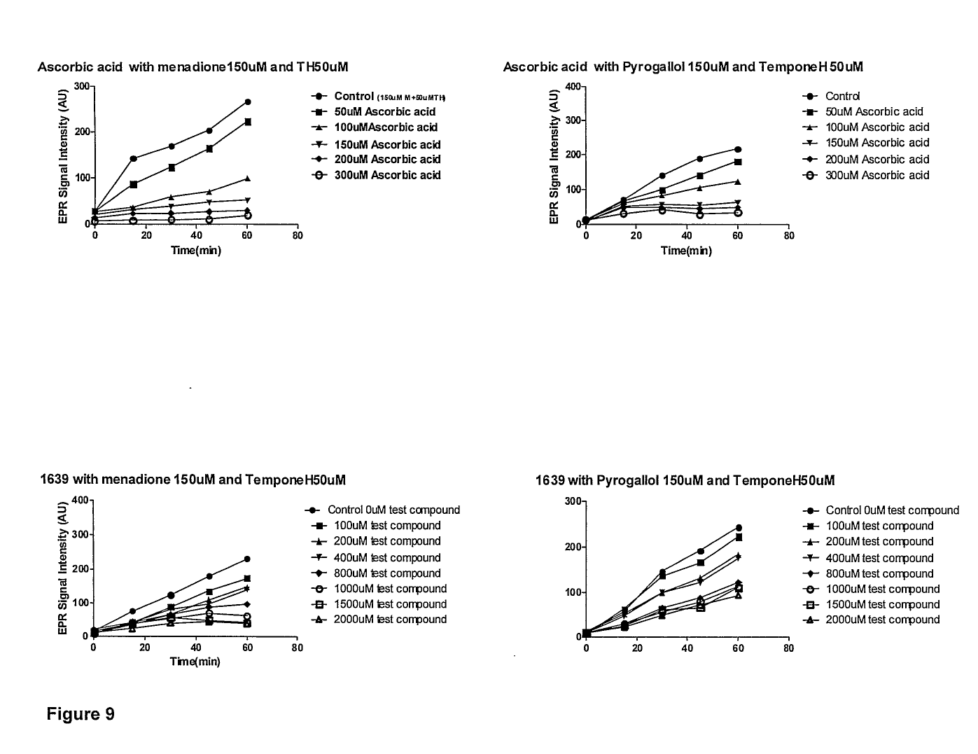

The ARE comparison was also carried out between p1639C (5-hexyl-2(5H)-furanone), 4-decanolide (5-hexyl-2(3H)-furanone) and 2(5H)-furanone. As can be seen in FIG. 8 compound p1639C showed an elicitation of ARE-driven gene expression up to 18-fold by treatment with 30 μM of p1639C. However, 4-decanolide or 2(5H)-furanone did not show significant anti-oxidant elicitation effects using ARE-driven gene expression methods.

A further antioxidant assay was conducted:

### Competitive EPR Antioxidant Assay

The basis of the assay is to assess the ability of different concentrations (100-2000 μM) of a test compound to compete with a standard concentration of spin trap (tempone-H; 50 μM) for hydroxyl or superoxide radicals. To our knowledge, this is the first assay that descriminates between scavenging capabilities for different oxygen-centred radicals.

In the absence of antioxidant, tempone-H reacts with oxygen-centred radicals to generate a spin signal at a rate that is determined by the concentration of the radical generating compound (menadione for .OH and pyrogallol for superoxide). Inclusion of an antioxidant with an equivalent rate constant for reaction with either OH or superoxide at the same concentration as tempone-H (50 μM) will effectively compete for radicals and will diminish the signal at a given timepoint by ˜50%. By monitoring the effect of a variety of different concentrations of test compound against a set concentration of tempone-H, a concentration-effect curve can be established, from which an IC₅₀ (concentration required to reduce the signal by 50% of control) can be derived for each test compound. Test compounds can then be compared to a known agent (in this case, ascorbic acid) and can be ranked according to scavenging capabilities for each of the radicals in question (.OH and O₂⁻). The lower the IC₅₀, the more potent the antioxidant.

### Results

Incubation (60 min, 37° C.) of tempone-H (50 μM) with menadione (150 μM; OH generator) or pyrogallol (150 μM O₂⁻ generator) caused a time-dependent increase in EPR signal (FIGS. 1a and b). Inclusion of ascorbic acid (50-300 μM) in the incubation mixture caused a concentration-dependent reduction in signal development (FIG. 1); 50% reduction in area under the curve was calculated to be ˜70 μM (IC₅₀) for .OH and 130 μM for O₂⁻. Equivalent experiments with test compound p1639C caused a less dramatic reduction in EPR signal (with a calculated IC₅₀ of ˜900 μM for OH experiments and ˜725 μM for O₂⁻.

### Interpretation

Compound p1639C has direct antioxidant effects against both .OH and O₂⁻ (to similar degrees) The potency of this agent is lower than that of ascorbic acid against both radical species (˜1 order of magnitude).

IV. Antifungal activity assay

Candida albicans and Malassezia furfur were used to carry out the antifungal assay in Potato Dextrose Agar (PDA) media containing 0.2% yeast extract. The Minimum Inhibitory Concentration (MIC) was recorded (Table 1).

TABLE 1
Comparison of antimicrobial activity of P1639C with some butenolides

| | | Double | C. albicans | M. furfur | MRSA |
|---|---|---|---|---|---|
| | R-group | bonds | (μg) | (μg) | (μg) |
| 2(5H)-furanone 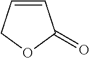 | C0 | C_{2, 3} | >10 | >10 | >20 |
| 5-methyl-2(3H)-furanone (α-angelica lactone, C1) 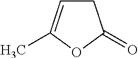 | Cl | C_{3, 4} | 5-7 | 5-7 | >20 |
| 5-methyl-2(5H)-furanone (C1) 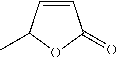 | Cl | C_{2, 3} | 5-7 | 5-7 | >20 |
| 3-methy1-2(5H)-furanone 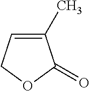 | Cl | C_{2, 3} | 5-7 | 5-6 | >20 |
| P1639C 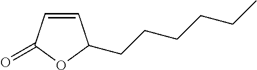 | C6 | C_{2, 3} | 1-1.5 | <1 | >10 |

P1639C showed antifungal activity against C. albicans at an MIC of 1-1.5 μg/ml and against M. furfur at 0.8 μg/ml. The comparison of antifungal activity using various butenolide compounds suggested that the length of the gamma side chain has significant effect on the anti-fungal activity. Results indicated that the longer the side chain, the better antifungal activity it possesses. However, considering the water solubility of the lactone compounds, a gamma side chain with carbon number between C5 and C8 is preferable. In regard to the antifungal activity, the double bond between C2 and C3 or C3 and C4 did not show significant effect on the activity.

### V. Anti-inflammatory Assay

Anti-inflammatory assay was carried out based on the NFκB expression and IKKβ activity. NFκB expression was tested using PRINCESS® NINA NFκB Assay Kit. P1639C and other related lactone compounds were supplemented to a mammalian cell culture in which NFκB has been stimulated by TNF-α. The assay was also coupled with an in vitro cell toxicity assay. IKKβ was tested using 5-20 mU of IKKβ, which was diluted in 50 mM Tris (pH 7.5), 0.1 mM EGTA, 1 mg/ml BSA, 0.1%, b-Mercaptoethanol. The kinase is assayed against substrate peptide (LDDRHDSGLDSMKDEEY) in a final volume of 25.5 μl containing 50 mM Tris (pH 7.5), 0.1 mM EGTA, 0.1%, b-Mercaptoethanol, 300 μM substrate peptide, 10 mM magnesium acetate and 0.005 mM [33P-g-ATP] (500-1000 cpm/pmole) and incubated for 30 mins at room temperature. Assays are stopped by addition of 50 of 0.5M (3%) orthophosphoric acid. The phosphorylated peptide was harvested on a p81 filterplate and the phosphorylation level was measured by scintillation counts.

P1639C was supplemented to the mammalian cell culture with a series of concentration at 0.001 mM, 0.002 mM, 0.005 mM, 0.01 mM, 0.02 mM, 0.05 mM, 0.1 mM, 0.2 mM and 0.5 mM. SEAP provided in the Princess NINA kit was used as a stimulatory control to show normal inflammatory response in cells, whereas BAY11-7082 at a final concentration of 5 μM was used as the positive control of inhibited NF-κB activity. The cell with TNF-α was used as the negative control. The cell response to the supplemented compounds was measured by the 450 nm emission (360 nm excitation) after addition of inactivation buffer and MUP solution.

Cell viability was carried out by incubation of the cells with resazurin and measuring the absorption at 600 nm against a reference measurement of above 650 nm.

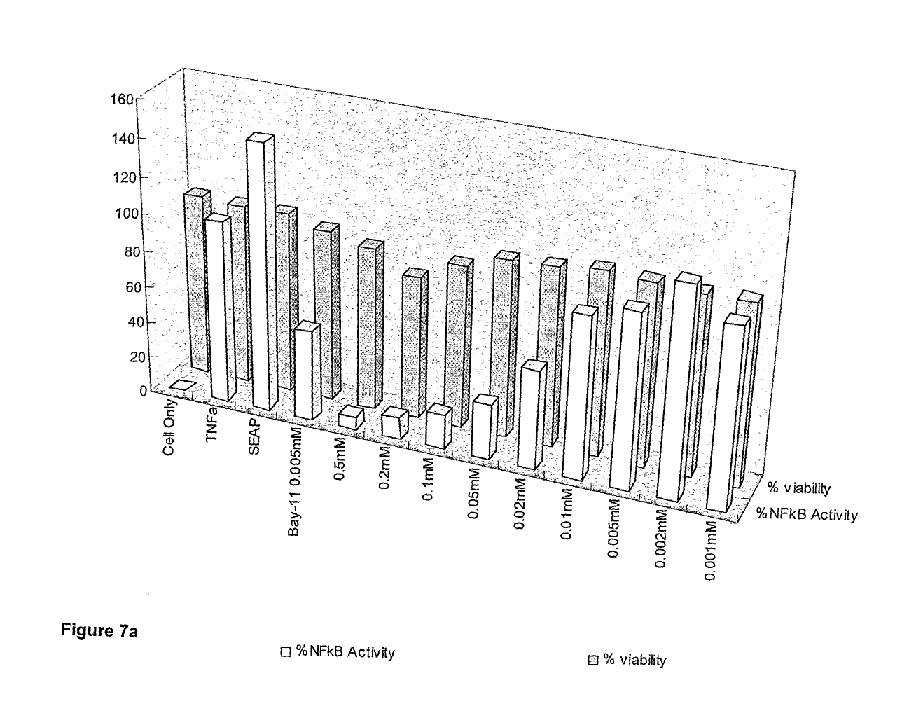

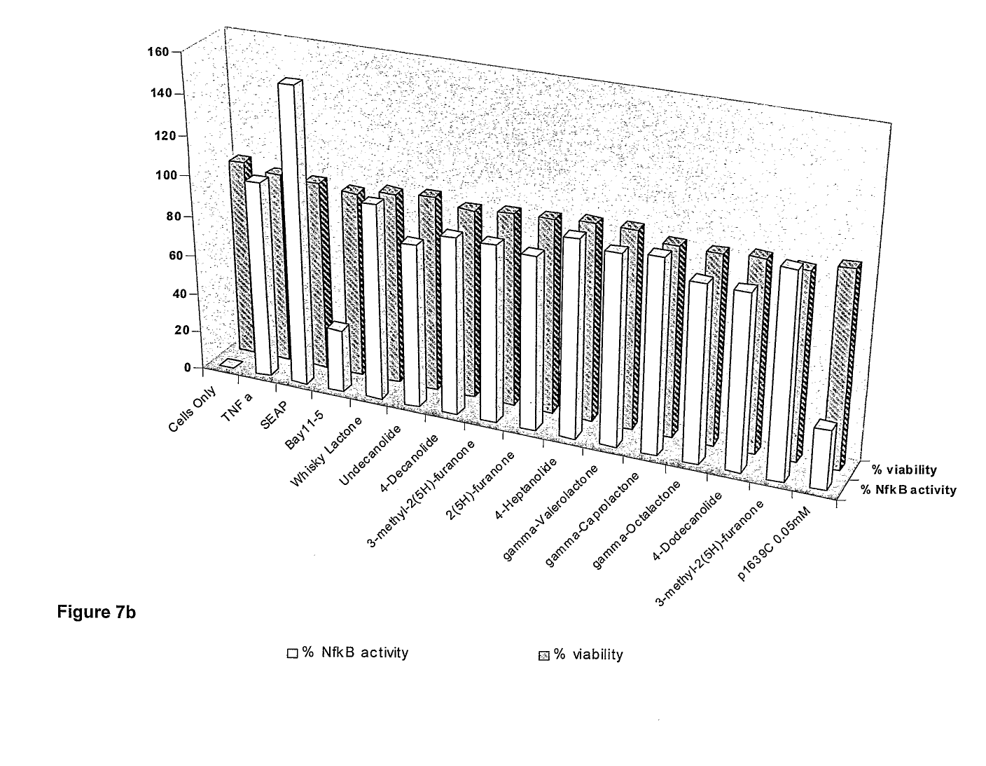

Lactone compounds sharing similar structure with p1639C were also examined for the anti-inflammatory activity. The comparison of single and double bonds with length of side chains were made between these lactones (FIGS. 7 a&b). In addition, IKKβ phosphorylation activity decreased to 64%±2% when 50 μM P1639C was present supporting the anti-inflammatory activity of P1639C.

### VI. Toxicology Study

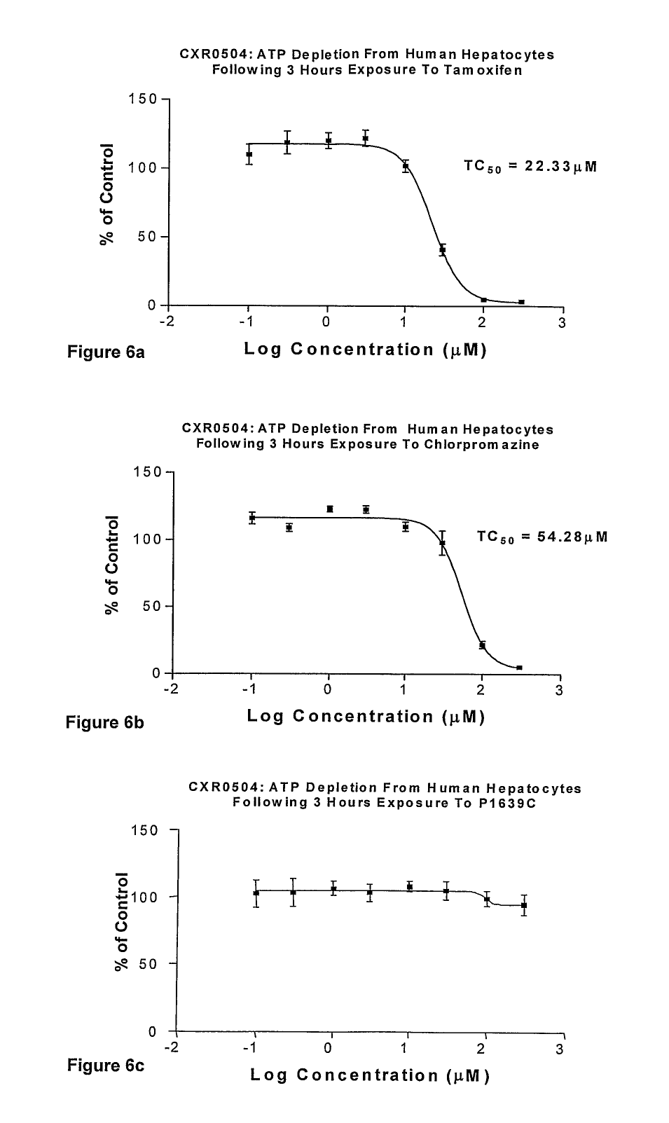

The toxicology assay was carried out using an in vitro human hepatocytes model where human hepatocytes were exposed to varying concentrations of compound P1639C as well as tamoxifen and chlorpromazine for three hours. The depletion rate of intracellular ATP was used as the parameter to measure the level of toxicity observed by each of the compounds. Both tamoxifen and chlorpromazine elicited a moderate decreasing rate of intracellular ATP level, which suggested a moderate level of toxicity. However, significant ATP depletion was not observed in human hepatocytes after exposure to compound P1639C, which suggested that the compound has low toxicity in human hepatocytes (FIGS. 6a-c).

Thus the applicant has found that in particular, the alkalinisation of the ethyl acetate extract from the AQP1639 culture supernatant generates a series of gamma-alkyl butenolides, in particular, a potent antifungal compound 5-hexylfuran-2(5H)-one, which also possesses an intense coconut fragrance, and strong antioxidant effects with low toxicity in human heptocyte toxicological models.

### REFERENCES

Braun, D., N. Pauli, et al. (1995). “New butenolides from the photoconductivity screening of Streptomyces antibioticus (Waksman and Woodruff) Waksman and Henrici 1948.” FEMS Microbiol Lett 126(1): 37-42.
Buttery, R. G. and L. C. Ling (1998). “Additional studies on flavor components of corn Tortilla chips.” J. Agric. Food Chem. 46(7): 2764-69.
Ding, G. J., P. A. Fischer, et al. (1998). “Characterization and quantitation of NF-kappaB nuclear translocation induced by interleukin-1 and tumor necrosis factor-alpha. Development and use of a high capacity fluorescence cytometric system.” J Biol Chem 273(44): 28897-905.
Franco, C. M., U. P. Borde, et al. (1991). “Butalactin, a new butanolide antibiotic. Taxonomy, fermentation, isolation and biological activity.” J Antibiot (Tokyo) 44(2): 225-31.
Hall, I. H., K. H. Lee, et al. (1979). “Anti-inflammatory activity of sesquiterpene lactones and related compounds.” J Pharm Sci 68(5): 537-42.
Herfarth, H., K. Brand, et al. (2000). “Nuclear factor-kappa B activity and intestinal inflammation in dextran sulphate sodium (DSS)-induced colitis in mice is suppressed by gliotoxin.” Clin Exp Immunol 120(1): 59-65.
Ikai, K., K. Takesako, et al. (1991). “Structure of aureobasidin A.” J Antibiot (Tokyo) 44(9): 925-33.
In, Y., T. Ishida, et al. (1999). “Unique molecular conformation of aureobasidin A, a highly amide N-methylated cyclic depsipeptide with potent antifungal activity: X-ray crystal structure and molecular modeling studies.” J Pept Res 53(5): 492-500.
Karin, M. and M. Delhase (2000). The I kappa B kinase (IKK) and NF-kappa B: key elements of proinflammatory signalling. Semin Immunol 12(1): 85-98.
Kato, J. Y., N. Funa, et al. (2007). “Biosynthesis of {gamma}-butyrolactone autoregulators that switch on secondary metabolism and morphological development in Streptomyces.” Proc Natl Acad Sci USA 104(7): 2378-83.
Kim, S. J., H. J. Jeong, et al. (2005). “Anti-inflammatory activity of gumiganghwaltang through the inhibition of nuclear factor-kappa B activation in peritoneal macrophages.” Biol Pharm Bull 28(2): 233-7.
Koch, E., C. A. Klaas, et al. (2001). “Inhibition of inflammatory cytokine production and lymphocyte proliferation by structurally different sesquiterpene lactones correlates with their effect on activation of NF-kappaB.” Biochem Pharmacol 62(6): 795-801.
Lin, Z. P., Y. C. Boller, et al. (1998). “Prevention of brefeldin A-induced resistance to teniposide by the proteasome inhibitor MG-132: involvement of NF-kappaB activation in drug resistance.” Cancer Res 58(14): 3059-65.
Lyss, G., A. Knorre, et al. (1998). “The anti-inflammatory sesquiterpene lactone helenalin inhibits the transcription factor NF-kappaB by directly targeting p65.” J Biol Chem 273(50): 33508-16.
Moussaieff, A., E. Shohami, et al. (2007). “Incensole Acetate, a Novel anti-inflammatory compound Isolated from Boswellia Resin, Inhibits Nuclear Factor (NF)-kappa B Activation.” Mol Pharmacol.
Nichols, T. C., T. H. Fischer, et al. (2001). “Role of nuclear factor-kappa B (NF-kappa B) in inflammation, periodontitis, and atherogenesis.” Ann Periodontol 6(1): 20-9.
Rouseff, R. L., M. M. Leahy, et al. (1995). Fruit flavors: biogenesis, characterization, and authentication. Washington, D.C., American Chemical Society.
Takano, E., T. Nihira, et al. (2000). “Purification and structural determination of SCB1, a gamma-butyrolactone that elicits antibiotic production in Streptomyces coelicolor A3(2).” J Biol Chem 275(15): 11010-6.
Yamamoto, Y., M. J. Yin, et al. (2000). IkappaB kinase alpha (IKKalpha) regulation of IKKbeta kinase activity. Mol Cell Biol 20(10): 3655-66.
Yoshikawa, Y., K. Ikai, et al. (1993). “Isolation, structures, and antifungal activities of new aureobasidins.” J Antibiot (Tokyo) 46(9): 1347-54.

1-29. (canceled)

30. A method of (a) treating a fungal infection, oxidant damage, bacterial infection, viral infection, inflammatory conditions and/or cancer or (b) preventing spoilage of, or presenting a food or beverage, said methods comprising administering or adding an effective amount of a compound according to formula (II) 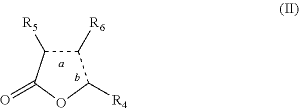
wherein,
R₄ is a C₁-C₁₂ alkyl group,
R₅ and R₆ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone,
the dashed lines a and b are independently single or double bonds, but not both together double bonds,
to a patient in need thereof or to a food or beverage.

31. The method according to claim 30, for the prevention or treatment of acne, dandruff and/or nail fungus.

32. The method of claim 30, wherein the compound is a compound according to formula (I), 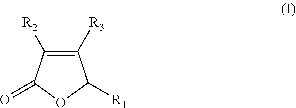
wherein,
R₁ is a C₆-C₁₂ alkyl group,
R₂ and R₃ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone.

33. The method according to claim 30, wherein R₄ is a C₆-C₁₂ alkyl group.

34. The method according to claim 30, wherein R₁ and R₄ are a C₆-C₁₀ alkyl group.

35. The method according to claim 34, wherein R₁ and R₄ are n-hexyl.

36. The method according to claim 30, for treating a Candida sp, Malassezia sp, Pityrosporum sp and/or Trichophyton sp. infection.

37. The method according to claim 30 wherein the compound according to formula (II) is in an amount of from 1 μg/ml to 100 μg/ml.

38. The method according to claim 37, wherein the compound according to formula (II) is in an amount of from 1 μg/ml to 10 μg/ml.

39. A method of adding a flavour or a fragrance to a food stuff, beverage, perfume or pharmaceutical product, comprising adding thereto a compound according to formula (I), 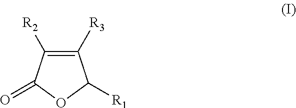
wherein,
R₁ is a C₆-C₁₂ alkyl group,
R₂ and R₃ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone.

40. The method according to claim 39 wherein the compound is provided as a component of a food and/or beverage.

41. A cosmetic preparation comprising a compound according to formula (II) 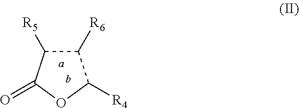
wherein,
R₄ is a C₁-C₁₂ alkyl group,
R₅ and R₆ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone,
the dashed lines a and b are independently single or double bonds, but not both together double bonds,
together with one or more carrier ingredients.

42. A cosmetic preparation according to claim 41, selected from skin crème, anti-ageing crème, skin toning crème, a skin-whitening preparation, skin wash, shampoo, hair conditioner, hair dye, pomade and hair mousse.

43. The method according to claim 30(b), wherein the compound is provided as an anti-spoilage and/or preservative component of food and/or beverage.

44. A method of preparing a compound according to formula (II) 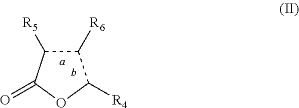
wherein,
R₄ is a C₁-C₁₂ alkyl group,
R₅ and R₆ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone,
the dashed lines a and b are independently single or double bonds, but not both together double bonds, comprising the steps:
i) providing an Aureobasidium sp. fungus, and culturing in a suitable culture medium under conditions and for an appropriate time period suitable for production of said compound or compound precursor; and
ii) recovering said compound, from the resultant culture.

45. A method according to claim 44, wherein R₄ is a C₆-C₁₂ alkyl group.

46. A method according to claim 44, wherein R₄ is n-hexyl.

47. A method according to claim 44, wherein R₅ and R₆ are independently hydrogen.

48. A method according to claim 44, wherein the dashed line “a” is a double bond, and the dashed line b is a single bond.

49. A method according to claim 44, wherein the Aureobasidium sp. fungus is a marine species.

50. A method according to claim 49, wherein the Aureobasidium sp. fungus is that which is deposited at CABI Bioscience UK Centre IMI and identified by accession number IMICC No. 394867, or a mutant or variant thereof.

51. A method according to claim 44, wherein said recovery step includes an alkalisation step.

52. A method according to claim 44, wherein said recovery step includes a solvent extraction step.

53. A method according to claim 44, wherein an amount of said compound according to formula (II) of at least greater than 5 mg/L of culture is obtained.

54. A method according to claim 53, wherein said obtained amount of compound is at least greater than 10 to 15 mg/L of culture.

55. The Aureobasidium sp. fungus which is deposited at CABI Bioscience UK Centre IMI and identified by accession number IMICC No. 394867, or a mutant or variant thereof.

56. A method of preparing a compound according to formula (II) 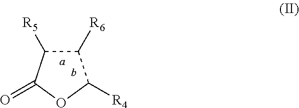
wherein,
R₄ is a C₁-C₁₂ alkyl group,
R₅ and R₆ are independently selected from H, alkyl, alkenyl, alkynyl, aryl, heteroaryl, carboxy, alkyloxycarbonyl, hydroxyl, amino, nitro, alkyloxy, alkylthio, formyl, cyano, carbamoyl, halo or a ketone,
the dashed lines a and b are independently single or double bonds, but not both together double bonds, comprising the steps:
i) providing a linear unsaturated fatty acid, and
ii) internally cyclising said fatty acid to provide said compound.

57. The method of claims 30, 41, 44 and 56, wherein the dashed line a is a double bond and the dashed line b is a single bond.


SEQUENCE LISTING (1 sequence)

SEQ ID NO: 1 - protein, 17 aa, Artificial Sequence
  Synthetic kinase substrate peptide
LDDRHDSGLDSMKDEEY